(19) United States
(12) Patent Application Publication
(10) Pub. No.: US 2003/0060775 A1
(43) Pub. Date: Mar. 27, 2003

(54) Disposable safety syringe

(76) Inventor: Mei-Ching Shyu, Taipei (TW)

Correspondence Address: Beehler & Pavitt, 100 Corporate Pointe, Suite 330, Culver City, CA, 90230, US

(21) Appl. No.: 09/967,209

(22) Filed: Sep. 27, 2001

(51) Int. Cl. A61M 5/32

(52) U.S. Cl. 604/197; 604/110

(57) ABSTRACT

A disposable safety syringe has a barrel, a connector assembled at a front end of the barrel, a needle assembly assembled onto the connector, and a plunger extendable into the barrel. The plunger has a connecting head such that the connecting head is able to be securely connected with the connector after an injection of the syringe. Therefore, the needle assembly is able to be pulled by the plunger to fully retract into the barrel and the syringe is then safe to discard without the risk of scratching someone.

### BACKGROUND OF THE INVENTION

1. Field of the Invention

The present invention relates to a syringe and more particularly to a disposable safety syringe. The syringe has a retractable needle to be pulled inside a barrel by a plunger after use, whereby accidental pricking is prevented.

2. Description of Related Art

Healthcare workers routinely have the risk of contracting communicable diseases through accidental exposure to contaminated medical waste products. One of the largest exposure risks to healthcare workers and handlers of the medical waste products is from accidental pricking from used syringe needles. It is reported that odds of a healthcare worker contracting human immunodeficiency virus (HIV) alone through accidental pricking are one in 300. The odds of healthcare workers contracting other serious diseases are even greater.

Therefore a current disposable syringe having a retractable needle is designed to solve the accidental pricking problem. The disposable syringe typically has a retractable needle which is able to be captured by a plunger. By pulling the plunger, the needle is fully retracted inside a barrel, and the contaminated syringe is able to be discarded safely without the risk of pricking someone.

However, the disposable syringe having the retractable needle is able to solve the accidental pricking problem only under an ideal situation. Further problems may still arise in practical use, for example, when retracting the needle, the connection between the needle and the plunger is not secure enough, and may undesirably detach before the needle is fully retracted inside the barrel. The undesirable detachment may cause a health risk to the user due to the chance of accidental pricking. Furthermore, before an injection can be given, air bubbles in the fluid contained in the syringe must be evacuated. For conventional syringes, the user usually has to point the needle upward and flick the barrel to help the air bubbles to evacuate. This procedure is not convenient and needs to be improved.

Accordingly, the present invention tends to provide a disposable safety syringe to mitigate or obviate the aforementioned problems.

### SUMMARY OF THE INVENTION

An objective of the present invention is to provide a disposable safety syringe to prevent accidental pricking, scratching or other harmful exposure to healthcare workers. The syringe has a barrel, a connector assembled at a front end of the barrel, a needle assembly fitted onto the connector, and a plunger extendable into the barrel. The plunger has a connecting head such that the connecting head is able to capture the connector after an injection process is completed. Therefore, the needle assembly is able to be pulled by the plunger to fully retract into the barrel and the syringe is then safe to discard without the risk of scratching someone.

Another objective of the present invention is to provide a disposable safety syringe having the structure as mentioned in the previous objective, wherein the connection between the connector and the connecting head of the plunger is secure and undesirable detachment of the connector and the connecting head during retraction of the needle assembly is prevented.

Another objective of the present invention is to provide a disposable safety syringe specially designed such that air bubbles in the fluid contained in the barrel before an injection are easily ejected prior to the injection process.

Other objectives, advantages and novel features of the invention will become more apparent from the following detailed description when taken in conjunction with the accompanying drawings.

### BRIEF DESCRIPTION OF THE DRAWINGS

FIG. 1 is an exploded, perspective view of a disposable safety syringe in accordance with the present invention;

FIG. 2 is a perspective view of the syringe in assembly;

FIG. 3 is a cross-sectional view of the syringe taken along line 3-3 in FIG. 2;

FIG. 4 is a partially enlarged, cross-sectional view showing a front portion of the syringe, wherein a connector head securely captures a connector;

FIG. 5 is a cross-sectional view of the syringe after use, wherein a needle assembly is retracted into a barrel and a plunger is broken off for convenient discarding; and

FIG. 6 is a partially enlarged, cross-sectional view showing the connector and the connecting head.

### DETAILED DESCRIPTION OF THE PREFERRED EMBODIMENT

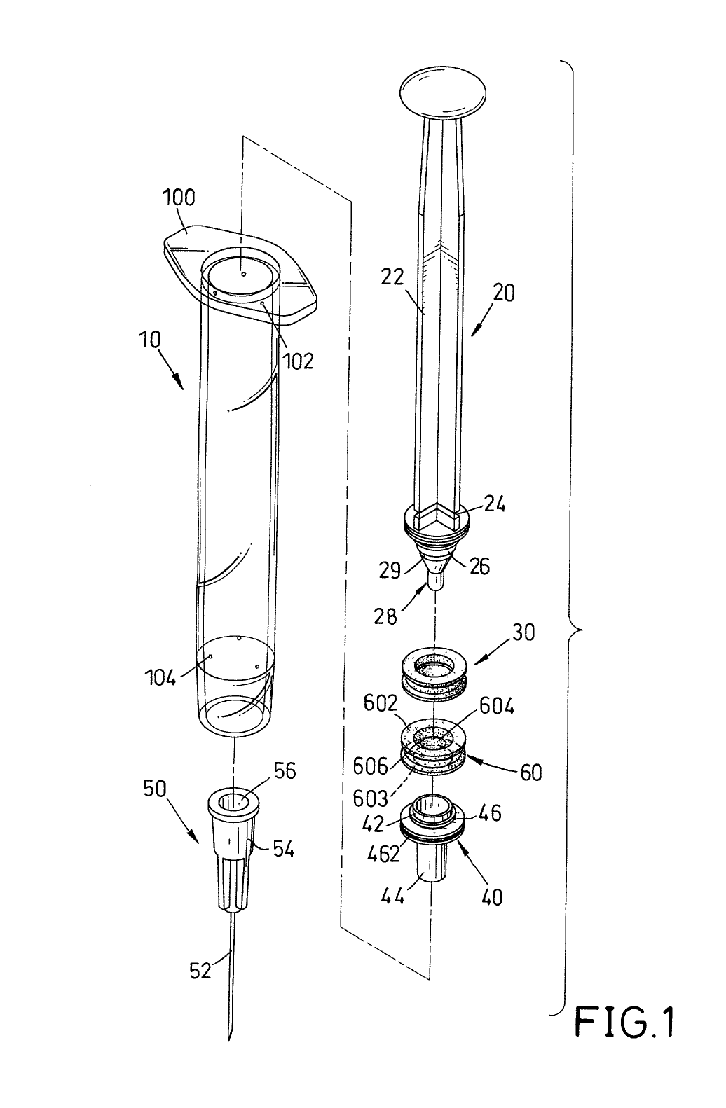

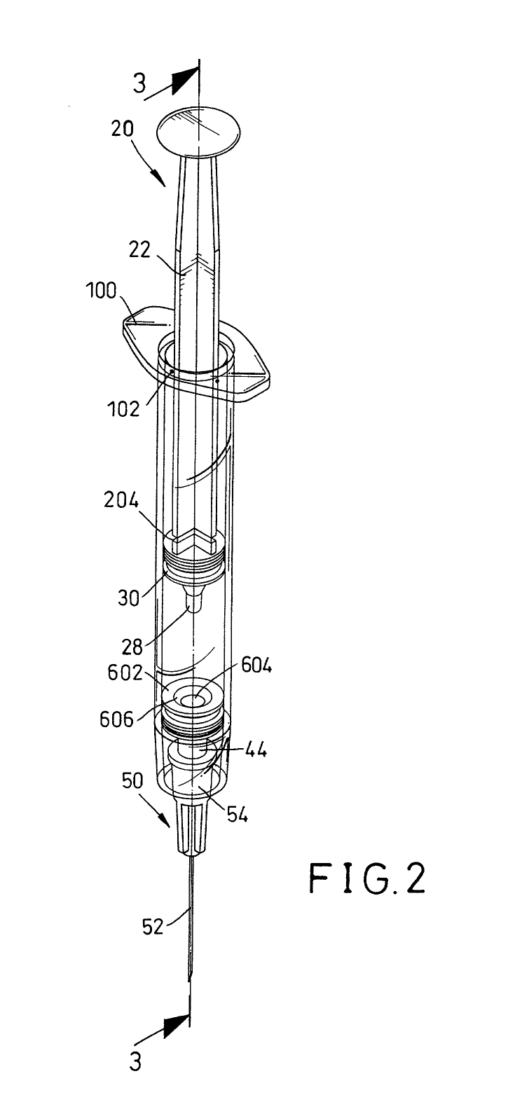

With reference to FIGS. 1 and 2, a disposable safety syringe in accordance with the present invention, which includes a barrel (10), a plunger (20), a connecting head (28), an upper sealing ring (30), a connector (40), a needle assembly (50), and a lower sealing ring (60).

The barrel (10) is a hollow cylinder having a front open end and a rear open end. A finger flange (100) is integrally formed at and extending outwardly from the rear open end. Multiple restricting protuberances (102) are formed on an inner surface of the barrel (10) close to the rear open end. Multiple retaining protuberances (104) are formed on the inner surface of the barrel (10) and close to the front open end.

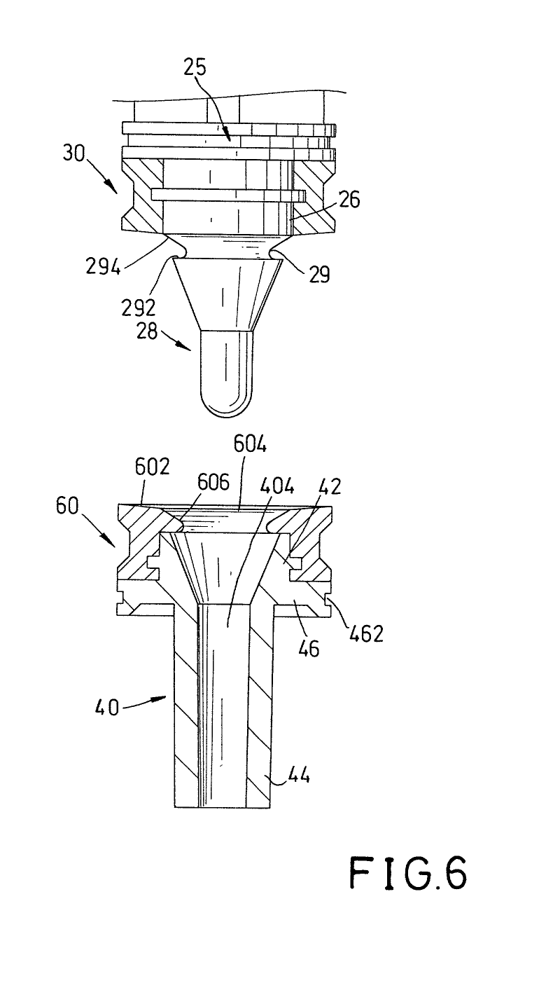

With reference to FIGS. 1 and 6, the plunger (20) is extendable into the barrel (10) and includes multiple elongated body pieces (22), a thumb pad formed at a rear end of the plunger (20) and a piston (25) formed at a front end of the plunger (20). Each one of the body pieces (22) is provided with a fracture line (24) defined near a front end of the plunger (20) so as allow the body pieces (22) to be easily broken off and thus separated from the piston (25) after completion of the injection process. The piston (25) comprises a retaining portion (26) for the upper sealing ring (30) to be mounted around. A connecting head (28) having a conical configuration is integrally formed with the retaining portion (26). An annular groove (29) is defined in the connecting head (28) and adjacent to the retaining portion (26). More specifically the annular groove (29) is defined by an abutting surface (292) and a back surface (294), wherein the back surface (294) is inclined toward a free end of the connecting head (28).

The connector (40) has a positioning portion (42) for the lower sealing ring (60) to mount around, a tube portion (44) for the needle assembly (50) to mount onto, and a disk portion (46) formed between the positioning portion (42) and the tube portion (44). A passage (404) is longitudinally defined through the connector (40). An annular slit (462) is defined around the disk portion (46) to receive the restricting protuberances (102) and thereby connects the connector (40) to the front opening end of the barrel (10).

The lower sealing ring (60) to be mounted around the positioning portion (42) of the connector (40) has a top surface (602), a bottom surface (603), and a bore (604) defined through the lower sealing ring (60) from the top surface (602) to the bottom surface (603). An annular flange (606) having a configuration corresponding to the annular groove (26) of the connecting head (28) integrally extends from the top surface (602) and into the bore (604) to be received in the annular groove (26) defined in the connecting head (28).

The needle assembly (50) to be mounted onto the connector (40) has a hollow needle (52) extending out from a needle hub (54), wherein the hollow needle (52) and the needle hub (54) are securely connected together. A receiving hole (56) configured for receiving the tube portion (44) of the connector (40) is longitudinally defined in the needle hub (54).

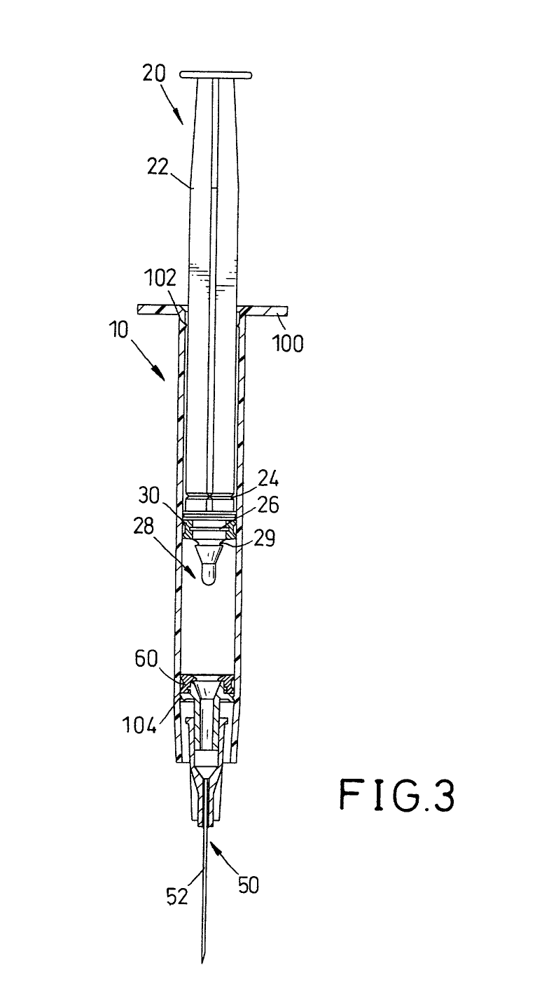

With reference to FIGS. 2 and 3, when assembling the disposable safety syringe, the lower sealing ring (60) is mounted around the positioning portion (42) of the connector (40), wherein the bore (604) defined in the lower sealing ring (60) communicates with the passage (404) defined in the connector (40). Then the connector (40) is assembled into the front open end of the barrel (10) by receiving the retaining protuberances (104) of the barrel (10) into the annular slit (462) of the disk portion (46) of the connector (40). Next the upper sealing ring (30) is mounted around the retaining portion (26) of the plunger (20), and the plunger (20) extends the connecting head (28) into the barrel (10) via the rear opening end of the barrel (10). Proper force is required to push the connecting head (28) to slide over the retaining protuberances (104) formed close to the rear open end of the barrel (10). Finally the needle assembly (50) is assembled onto the connector (40) by force fitting the tube portion (44) of the connector (40) into the receiving hole (56) defined in the needle assembly (50). At this moment, the syringe is ready to be used. The procedure of performing an injection using the syringe is same as those of the prior art syringes, and owing to that syringes are widely known medical instruments and have been used for years, therefore excessive description on the injection procedure of the syringe is omitted. However, it is noted that due to the conical configuration of the connecting head (28) and that the back surface (294) is inclined toward a free end of the connecting head (28), air bubbles in the fluid contained in the barrel (10) are easily ejected by pointing the needle (52) upward and pushing the plunger (20) a little further into the barrel (10). The filliping against the barrel (10) to help the air bubbles be ejected as usually required before use of a conventional syringe is not necessary.

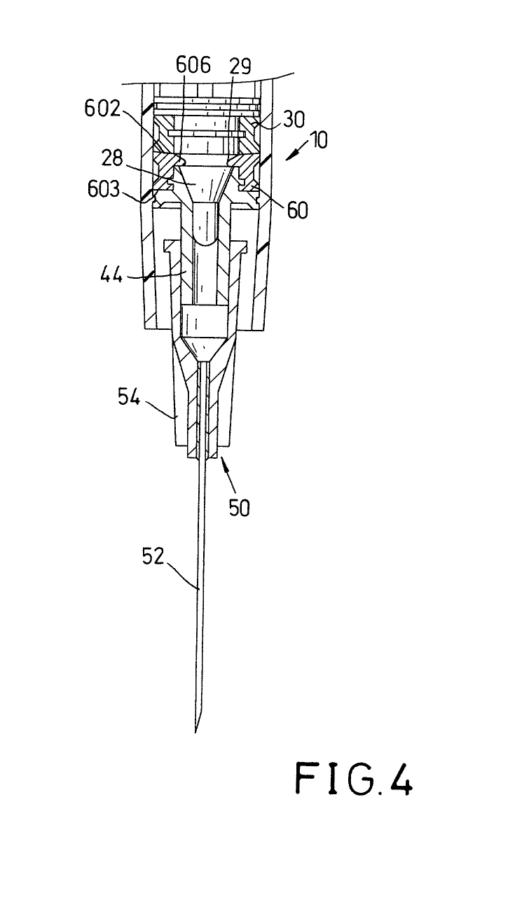

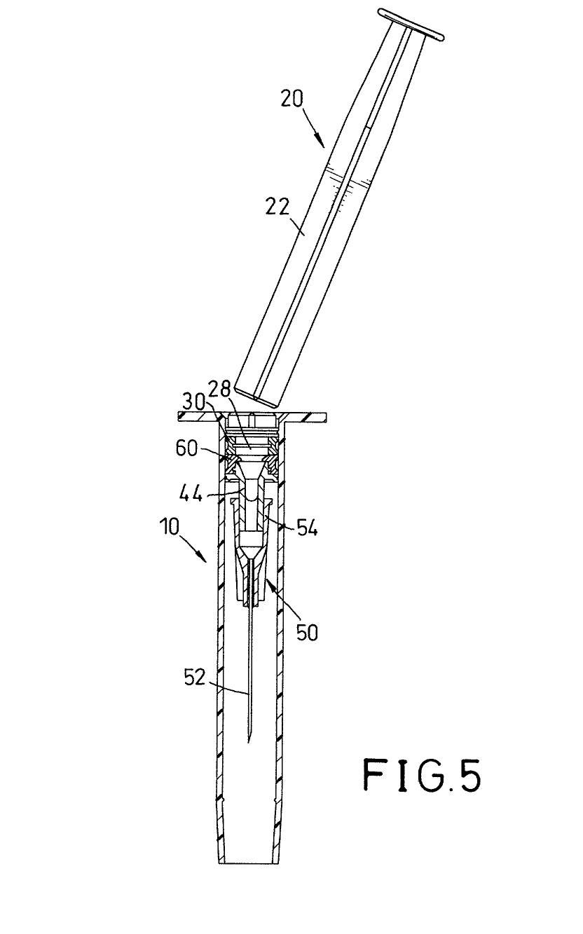

With reference to FIG. 4, after use of the syringe in accordance with the present invention, the plunger (20) is pushed further into the barrel (10) so as to allow the connecting head (28) to be securely connected with the connector (40) by securely receiving the annular flange (606) of the upper sealing ring (60) in the corresponding annular groove (29) defined in the connecting head (28). The plunger (20) is then pulled so as to pull the connector (40) together with the needle assembly (50) to retract into the barrel (10). With reference to FIG. 5, the needle assembly (50) is pulled until the needle (52) is fully retracted inside the barrel (10) and the restricting protuberances (102) (better seen in FIG. 2) prevent the connecting head (28) from escaping from the rear open end of the barrel (10). The piston (25) is separated by the body pieces (22) of the plunger (20) being broken off along the fracture lines (24). Therefore, the contaminated needle (52) is received inside the barrel (10) and the used syringe is able to be discarded safely without accidentally scratching someone.

From the above description, it is noted that the invention has the following advantages:

1. The connection between the connecting head (28) and the lower sealing ring (60) is secure and undesirable detachment during the retraction of the needle assembly (50) is prevented.

2. The connecting head (28) and the lower sealing ring (60) have specially designed configurations such that the air bubbles are easily ejected from the barrel (10) before an injection is given.

3. The structure of the syringe is simple and suitable for mass production.

While this invention has been particularly shown and described with references to the preferred embodiment thereof, it will be understood by those skilled in the art that various changes in form and details may be made therein without departing from the scope of the invention encompassed by the appended claims.

What is claimed is:

1. A disposable safety syringe comprising:
a hollow barrel having a front open end and a rear open end;
a plunger extendable into the barrel from the rear open end, the plunger having a front end to extend into the barrel and an upper sealing ring securely mounted around the front end;
a connecting head formed at the front end of the plunger; the connecting head defining a groove;
a hollow connector assembled in the front open end of the plunger, the hollow connector having a positioning portion and a tube portion;
a lower sealing ring securely mounted around the positioning portion of the hollow connector, the lower sealing ring defining a bore extending through the lower sealing ring and having a flange extending into the bore to be received in the groove defined in the connecting head;
a needle assembly mounted onto the connector and having a needle hub and a needle extending out from the needle hub, the needle hub defining a receiving hole configured to allow the tube portion to be force fitted inside the receiving hole,
whereby after an injection process using the disposable safety syringe is completed, the connecting head and the lower sealing ring are able to be securely connected together by securely receiving the flange inside the groove, and therefore the needle assembly is able to be fully retracted inside the barrel by pulling the plunger such that the needle is safely received inside the barrel to prevent accidentally scratching someone.

2. The disposable safety syringe as claimed in claim 1, wherein the flange of the lower sealing ring is formed as an annular flange.

3. The disposable safety syringe as claimed in claim 2, wherein the groove defined in the connecting head is defined as an annular groove to correspond to the annular flange.

4. The disposable safety syringe as claimed in claim 1, wherein the flange is inclined toward a free end of the connecting head and the groove is defined corresponding to an inclined configuration of the flange to ensure the flange to be securely received in the groove.

5. The disposable safety syringe as claimed in claim 3, wherein the annular flange is inclined toward a free end of the connecting head and extends into the bore of the lower sealing ring, the annular groove is defined corresponding to an inclined configuration of the annular flange to ensure the annular flange to be securely received in the annular groove.
